(19) United States
(12) Patent Application Publication
(10) Pub. No.: US 2020/0370111 A1
(43) Pub. Date: Nov. 26, 2020

(54) MOLECULAR TAGGING SYSTEM WITH NANOPORE-ORTHOGONAL DNA BARCODES

(71) Applicant: University of Washington, Seattle, WA (US)

(72) Inventors: Luis H. Ceze, Seattle, WA (US); Kathryn J. Doroschak, Seattle, WA (US); Jeffrey M. Nivala, Seattle, WA (US)

(73) Assignee: University of Washington, Seattle, WA (US)

(21) Appl. No.: 16/879,214

(22) Filed: May 20, 2020

Related U.S. Application Data
(60) Provisional application No. 62/850,407, filed on May 20, 2019.

(51) Int. Cl. C12Q 1/6869; G16B 30/00; C12Q 1/6806; G06N 20/00

(52) U.S. Cl. CPC C12Q 1/6869; G06N 20/00; C12Q 1/6806; G16B 30/00

(57) ABSTRACT

In some embodiments, a molecular tagging system that uses synthetic DNA-based tags is provided. In some embodiments, a kit for tagging objects with molecular tags is provided that comprises a plurality of molbit reservoirs. Each molbit reservoir is associated with a molbit and includes nucleic acid molecules that represent the molbit. In some embodiments, a method is provided wherein a digital tag value is determined, the digital tag value is converted to a molbit tag value, nucleic acid molecules associated with each molbit value indicated as present in the molbit tag value are combined, and the combined nucleic acid molecules are applied to an object to tag the object. In some embodiments, a system is provided that includes a computing system configured to receive raw nanopore signals from a sequencing device, to identify molbits based on the signals, and to determine a digital tag based on the identified molbits.

### CROSS-REFERENCE TO RELATED APPLICATIONS

This application claims the benefit of Provisional Application No. 62/850,407, filed May 20, 2019, the entire disclosure of which is hereby incorporated by reference for all purposes.

### STATEMENT OF GOVERNMENT LICENSE RIGHTS

This invention was made with government support under Grant No. W911NF-18-2-0034, awarded by the Defense Advanced Research Projects Agency, and Grant No. CCF1518703, awarded by the National Science Foundation. The government has certain rights in the invention.

### BACKGROUND

Molecular tagging uses physical molecules to encode an identifier for a physical object. The basic objective of molecular tagging is to record an ID in a molecular format, attach it to an object, and then later be able to read out the identifier quickly and accurately. Applications for such a system include secret exchange and transfer, such as encryption keys and digital wallets; tracking and provenance; and counterfeit detection, including verifying a material's source from production through final receipt and use. Currently, no molecular tagging method exists that is inexpensive, fast and reliable to decode, and usable outside a lab setting to create or read tags.

### BRIEF SUMMARY

In some embodiments, a system comprising a sequencing device, a plurality of nucleic acid molecules, and a computing system is provided. The sequencing device is configured to generate signals based upon sequences of nucleic acid molecules. In the plurality of nucleic acid molecules, each nucleic acid molecule includes a barcode sequence associated with a molbit of a set of molbits. The computing system is communicatively coupled to the sequencing device and includes logic that, in response to execution by at least one processor of the computing system, causes the computing system to perform actions for determining a digital tag represented by the plurality of nucleic acid molecules, the actions comprising: receiving, from the sequencing device, a plurality of signals, wherein signals of the plurality of signals represent raw nanopore signals detected by the sequencing device while sequencing at least the barcode sequences of the plurality of nucleic acid molecules; identifying molbits represented by at least the barcode sequences of the plurality of nucleic acid molecules based on the plurality of signals; and determining a digital tag based on the identified molbits.

In some embodiments, a kit for tagging objects with molecular tags that represent digital tag values is provided. The kit comprises a plurality of molbit reservoirs, wherein each molbit reservoir is associated with a molbit and includes nucleic acid molecules that represent the molbit.

In some embodiments, a method of tagging an object with a molecular tag is provided. A digital tag value is determined. The digital tag value is converted to a molbit tag value, the molbit tag value indicating presence and absence of a plurality of molbit values. Nucleic acid molecules associated with each molbit value indicated as present in the molbit tag value are combined. The combined nucleic acid molecules are applied to the object.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a schematic diagram that illustrates a molbit design scheme according to various aspects of the present disclosure.

FIG. 2 is an illustration of a non-limiting example embodiment of a portion of a barcode sequence and the resulting ionic current nanopore trace, or “squiggle,” according to various aspects of the present disclosure.

FIG. 3 illustrates a non-limiting example embodiment of a system for processing molecular tags according to various aspects of the present disclosure.

FIG. 4 is a flowchart that illustrates a non-limiting example embodiment of a method of determining sequences for a set of molbits according to various aspects of the present disclosure.

FIG. 5 is a chart that illustrates further aspects of a non-limiting example embodiment of the method described in FIG. 4.

FIG. 6 is an illustration of a non-limiting example embodiment of a machine learning model suitable for use in identifying molbits according to various aspects of the present disclosure.

FIG. 7 is a flowchart that illustrates a non-limiting example embodiment of a method for training a machine learning model to identify molbits from sequence signal data according to various aspects of the present disclosure.

FIG. 8 is a flowchart that illustrates a non-limiting example embodiment of a method of using a molecular tag to label an object according to various aspects of the present disclosure.

FIG. 9 is a chart that illustrates tallies of various molbits identified by the machine learning model according to various aspects of the present disclosure.

FIG. 10 is a block diagram that illustrates a non-limiting example embodiment of a computing device appropriate for use as a computing device with embodiments of the present disclosure.

### DETAILED DESCRIPTION

DNA-based information storage continues to gain momentum with the emergence of high-throughput DNA sequencing (sequencing-by-synthesis) and synthesis (array-based synthesis). These technologies enable vast amounts of text and visual information to be encoded, stored, and decoded. DNA data storage offers unique advantages over mainstream storage methods like magnetic tape and hard disk drives, including higher physical density and longer retention lifetimes. This is particularly useful for archival storage, where current access speeds and read/write costs are not an issue. The development of portable, real-time sequencing (such as nanopore sequencing), together with new methods that simplify the modular assembly of pre-defined DNA sequences, creates additional opportunities for rapid writing and on-demand readout.

These advances can help improve an early application of DNA-based information storage: molecular tagging. Molecular tagging uses physical molecules to encode an identifier for a physical object, analogous to a radio-frequency identification (RFID) tag or two-dimensional barcode (e.g., a QR code) in the digital world. An ideal molecular tagging system should be inexpensive and reliable, with fast readout and user-controlled encoding and decoding from end-to-end. Such molecular tags could be easily used in situations where RFID tags and QR codes are not suitable. Some examples of appropriate situations include, but are not limited to, labeling and tracking commodities that are too small, flexible, or numerous to attach a sticker or electronic tag (especially to detect counterfeits); tracking and establishing provenance for higher value items; and covertly exchanging private information like encryption keys and digital wallets. Although several molecular tagging solutions exist, none is fully usable—from creation to readout—outside of a laboratory setting, or without involving a third party. These constraints significantly increase tag read and write latency, decrease tag confidentiality, and ultimately limit custom application development.

To address these (and other) issues, embodiments of the present disclosure provide a molecular tagging system that uses synthetic DNA-based tags. Although DNA is typically considered expensive for reading and writing, some embodiments of the present disclosure lower the cost and time delay involved in creating DNA-based molecular tags by providing a fixed library of pre-synthesized, nanopore-orthogonal DNA fragments, referred to herein as molecular bits (molbits). Molbits are used to represent digital values through presence or absence instead of as digital 1s and 0s. This allows new, arbitrary tags to be created by end-users for custom applications by simply mixing molbits to produce a molecular tag.

In some embodiments, such molecular tags may be read out quickly using a portable, low-cost sequencing device (such as the MinION device provided by Oxford Nanopore Technologies). In using sequencing devices, raw nanopore signals are typically converted back to a DNA sequence in a computationally expensive process and error-prone process called basecalling. To increase reading speed and accuracy, embodiments of the present disclosure are configured to classify molecular tags directly from raw nanopore signals, eliminating the need for basecalling. In some embodiments of the present disclosure, the number of molecular tags available are greatly increased by custom designing barcode sequences for the molbits that produce easily distinguishable ionic current signatures. In some embodiments, error correction values are also added to the tag to resolve decoding errors.

In some embodiments, molbits are prepared for readout (sequencing) prior to tag creation/application, and can be stabilized by dehydration to extend tag shelf life, decrease decoding time, and reduce contamination from environmental DNA. The result is a highly accurate real-time tagging system that includes a novel approach to developing nanopore-orthogonal barcodes. These barcodes, and the methods described herein to develop them, are extensible: they can be used both within systems as disclosed herein to tag physical objects, as well as beyond such systems for other molecule-level tagging needs like sample multiplexing for nanopore sequencing.

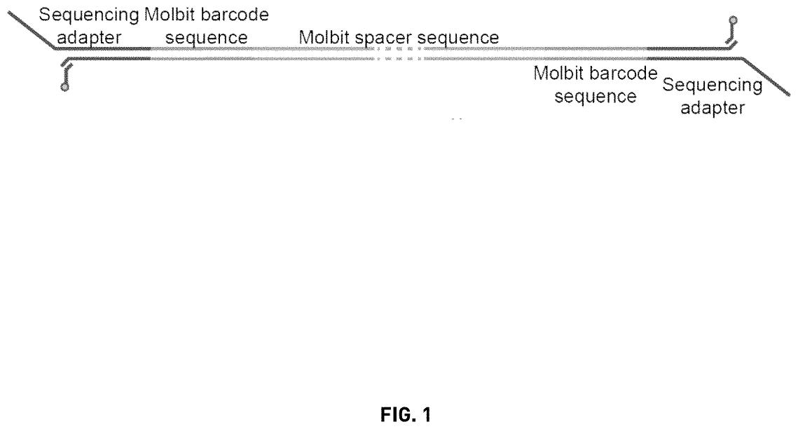

FIG. 1 is a schematic diagram that illustrates a molbit design scheme according to various aspects of the present disclosure. As shown, the molecule includes a sequencing adaptor, a barcode sequence, and a spacer sequence. The barcode sequence is attached to a spacer sequence using any suitable technique, including but not limited to Golden Gate assembly. In some embodiments, the barcode sequence may be made compatible with Golden Gate assembly by incorporating a short single-stranded overhang. The barcode sequence is attached to both ends of the spacer sequence and a sequencing adapter is attached to both ends of the molecule, thus allowing the barcode sequence to be sensed from either direction.

The spacer sequence is included to make the overall molecule of a sufficient length to be read by a sequencing device, and to provide an additional encoding channel. That is, in some embodiments, spacer sequences of different lengths may be used with the same barcode sequence, such that the barcode sequence may be used to represent more than one molbit value by being paired with a different length spacer sequence. For example, spacer lengths of about 400 nucleotides and about 1600 nucleotides may be used, and simple signal length binning may be sufficient for decoding. In one example test, the median signal length for the 400 and 1600 nucleotide strands was 5,768 and 16,968, respectively, and a cutoff at 9,800 provided 91% accuracy (with most errors caused by long strands misidentified as short strands).

The spacer technique can be used to double the number of molbits without significantly increasing read complexity, because with properly chosen spacer sequence lengths, the length of each read can be used to determine whether it represents a long spacer sequence or a short spacer sequence. Further details regarding the construction and use of molbit molecules are provided below.

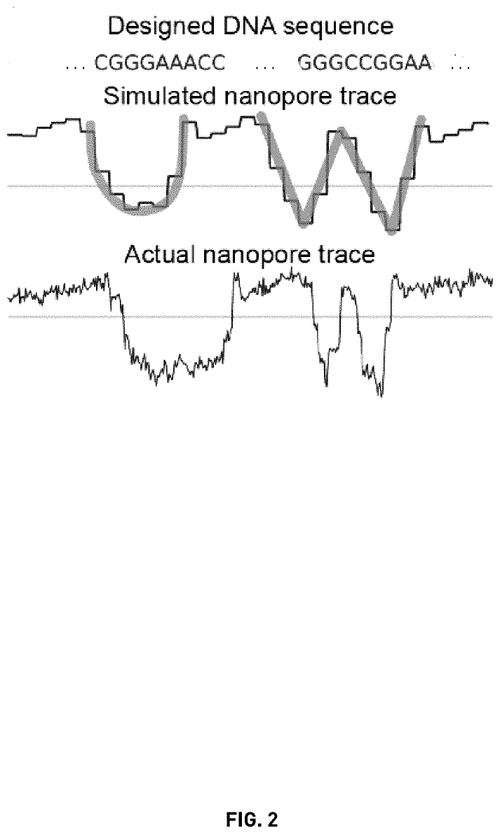

In some embodiments, in order to increase classification accuracy and decrease computation time, the barcode sequences used are designed to be identifiable from amongst each other without the use of basecalling. That is, the barcode sequences are designed to generate distinguishable ionic current signatures, sometimes referred to as “squiggles,” to promote unambiguous classification. FIG. 2 is an illustration of a non-limiting example embodiment of a portion of a barcode sequence and the resulting ionic current nanopore trace, or “squiggle,” according to various aspects of the present disclosure. Any suitable technique may be used to predict the ionic current signature generated by the barcode sequences. In FIG. 2, a result generated by Scrappie squiggler, a publicly available tool created by the Oxford Nanopore Research Algorithms group that converts sequences of bases to ionic current via a convolutional model, is shown. To demonstrate the ability of Scrappie to accurately model real nanopore squiggles, a barcode sequence was designed that appears as the letters “UW” in the simulated nanopore trace generated by Scrappie. As shown, the simulated trace has a high level of visual similarity with an actual nanopore trace of the designed barcode sequence.

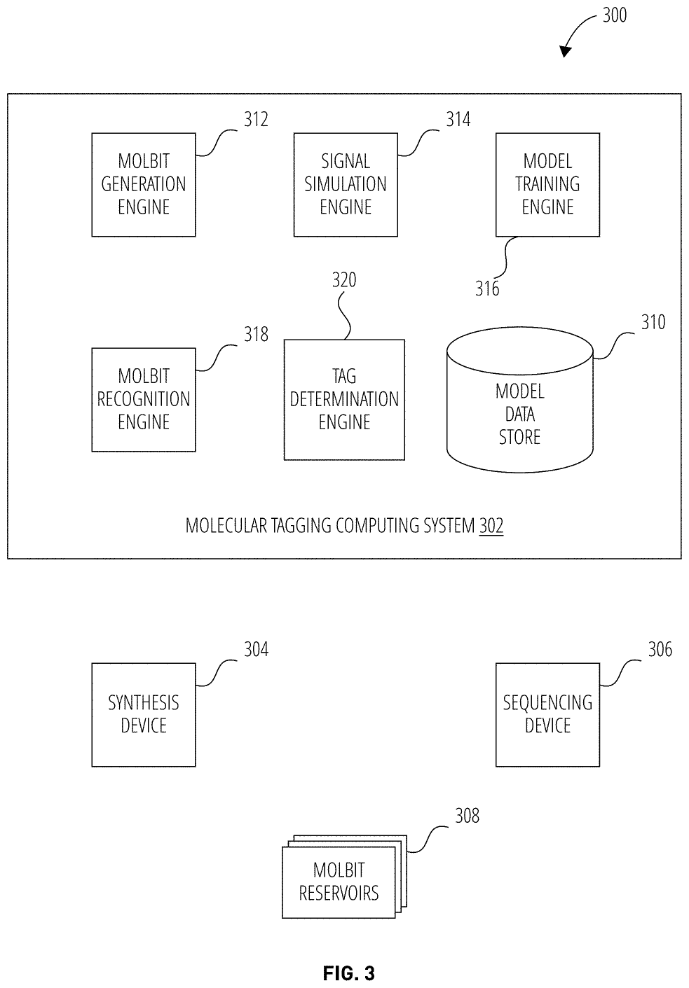

FIG. 3 illustrates a non-limiting example embodiment of a system for processing molecular tags according to various aspects of the present disclosure. The illustrated system 300 shows components for the determination of barcode sequences to be associated with molbits, the synthesis and storage of nucleic acid molecules that represent the molbits, the building of molecular tags made up of molbits, and the reading of molecular tags. In some embodiments, additional components may be included in the system 300, including but not limited to devices for lyophilization, devices for rehydrating molecular tags, and the like. Because the existence and use of these components are known to one of ordinary skill in the art, they have not been illustrated in FIG. 3 or described in detail for the sake of brevity.

As shown, the system 300 includes a molecular tagging computing system 302, a synthesis device 304, a sequencing device 306, and a plurality of molbit reservoirs 308.

In some embodiments, the synthesis device 304 may be any suitable device or collection of devices for creating molbit molecules. In some embodiments, the synthesis device 304 may include one or more computer-controlled oligonucleotide synthesizers technically implemented in column, multi-well plate, or array formats. In some non-limiting embodiments, the synthesis device 304 may be provided by a party such as Integrated DNA Technologies, Inc.

In some embodiments, the sequencing device 306 may be any suitable device or collection of devices for sequencing molbit molecules. In some embodiments, the sequencing device 306 may be selected to be portable and low-cost in order to increase the usefulness of the overall system 300. In some embodiments, the sequencing device 306 may be a nanopore sequencing device. One non-limiting example of a sequencing device 306 includes a MinION device provided by Oxford Nanopore Technologies, though other sequencing devices may be used.

In some embodiments, the molbit reservoirs 308 include a plurality of separate containers, each of which contains molecules that represent a separate molbit. By including a plurality of separate molbit reservoirs 308, molbits can be combined to form molecular tags simply by extracting molbits from the proper molbit reservoirs 308 associated with the molbits desired for the molecular tags. The molbit reservoirs 308 may be any suitable container for holding the molbit molecules.

In some embodiments, the molecular tagging computing system 302 may include one or more computing devices configured to provide the illustrated functionality. As some non-limiting examples, the molecular tagging computing system 302 may include one or more desktop computing devices, laptop computing devices, server computing devices, rack-mounted computing devices, and/or computing devices that are part of a cloud computing system. In some embodiments, the molecular tagging computing system 302 may be communicatively coupled to the sequencing device 306 and/or the synthesis device 304 using any suitable technique, including but not limited to wired communication technologies (including but not limited to Ethernet, USB, and FireWire), wireless communication technologies (including but not limited to 2G, 3G, 4G, 5G, LTE, Wi-Fi, WiMAX, and Bluetooth), exchange of physical computer-readable media (including but not limited to magnetic hard drives, optical disks, and flash memory), or combinations thereof.

As shown, the molecular tagging computing system 302 includes a molbit generation engine 312, a signal simulation engine 314, a model training engine 316, a molbit recognition engine 318, a tag determination engine 320, and a model data store 310.

In some embodiments, the molbit generation engine 312 is configured to determine barcode sequences that can be reliably distinguished from each other using raw nanopore trace signals from the sequencing device 306. In some embodiments, the signal simulation engine 314 is configured to simulate the raw nanopore trace signals that would be generated by the sequencing device 306 if it were to be processing a given sequence.

In some embodiments, the model training engine 316 is configured to use training data generated by the sequencing device 306 for a training set of molbit molecules synthesized by the synthesis device 304 to train a machine learning model to identify the molbit molecules using the raw nanopore trace signals generated by the sequencing device 306, and to store the machine learning model in the model data store 310. In some embodiments, the molbit recognition engine 318 is configured to retrieve the machine learning model from the model data store 310 and to use it to identify molbit molecules in molecular tags. In some embodiments, the tag determination engine 320 is configured to both translate digital tags in a digital address space into molecular tags in a molbit address space, but also to translate molecular tags in the molbit address space back into digital tags in the digital address space.

Further description of the actions performed by each of these components is provided below.

As used herein, “engine” refers to logic embodied in hardware or software instructions, which can be written in one or more programming languages, including but not limited to C, C++, C#, COBOL, JAVA™, PHP, Perl, HTML, CSS, JavaScript, VBScript, ASPX, Go, and Python. An engine may be compiled into executable programs or written in interpreted programming languages. Software engines may be callable from other engines or from themselves. Generally, the engines described herein refer to logical modules that can be merged with other engines, or can be divided into sub-engines. The engines can be implemented by logic stored in any type of computer-readable medium or computer storage device and be stored on and executed by one or more general purpose computers, thus creating a special purpose computer configured to provide the engine or the functionality thereof. The engines can be implemented by logic programmed into an application-specific integrated circuit (ASIC), a field-programmable gate array (FPGA), or another hardware device.

As used herein, “data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

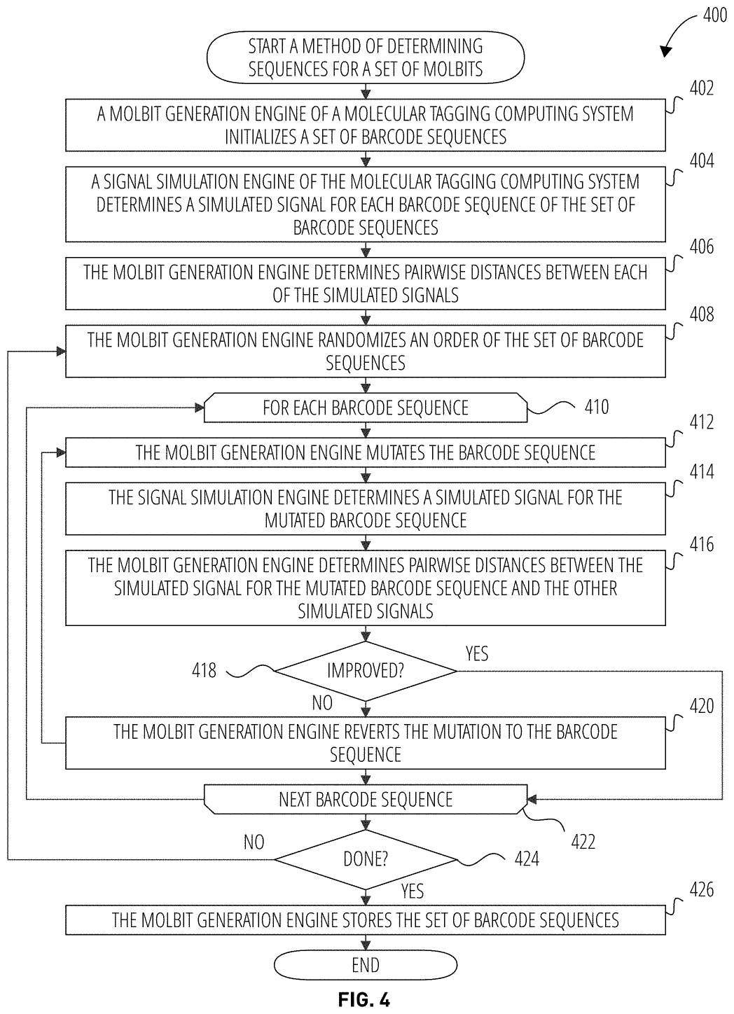

FIG. 4 is a flowchart that illustrates a non-limiting example embodiment of a method of determining sequences for a set of molbits according to various aspects of the present disclosure. As mentioned above, each molbit molecule includes a barcode sequence. This barcode sequence is designed in order to cause a sequencing device 306 to generate a signal that can be uniquely identified compared to the signals generated by the other barcode sequences with a high degree of reliability without requiring the use of basecalling by the sequencing device 306. The method 400 is a non-limiting example of one technique for determining such sequences.

From a start block, the method 400 proceeds to block 402, where a molbit generation engine 312 of a molecular tagging computing system 302 initializes a set of barcode sequences. The set of barcode sequences may include any appropriate number of barcode sequences. In some embodiments, 96 barcode sequences may be used, while in other embodiments, more or fewer barcode sequences may be used. Any suitable technique may be used to initialize the barcode sequences in the set of barcode sequences, including but not limited to randomizing the barcode sequences and assigning a set of pre-seeded starting barcode sequences (such as a set of barcode sequences from a previous iteration of the method 400 or another method).

At block 404, a signal simulation engine 314 of the molecular tagging computing system 302 determines a simulated signal for each barcode sequence of the set of barcode sequences. Any suitable technique may be used to determine the simulated signal. In some embodiments, the Scrappie squiggler tool may be used to determine the simulated signals.

At block 406, the molbit generation engine 312 determines pairwise distances between each of the simulated signals. Any appropriate technique may be used for determining the pairwise distances. In some embodiments, dynamic time warping (DTW) may be used as the measure of pairwise distance.

At block 408, the molbit generation engine 312 randomizes an order of the set of barcode sequences. The method 400 then proceeds to a for loop defined between for-loop start block 410 and for-loop end block 422, wherein each barcode sequence of the set of barcode sequences is processed in the random order established at block 408. Within the for-loop, each barcode sequence is individually mutated in an attempt to make each barcode sequence incrementally more different from the other barcode sequences as described below.

From the for-loop start block 410, the method 400 proceeds to block 412, where the molbit generation engine 312 mutates the barcode sequence. In some embodiments, a mutation may be introduced by simultaneously modifying two adjacent nucleotides in a random location.

At block 414, the signal simulation engine 314 determines a simulated signal for the mutated barcode sequence. In some embodiments, a technique that matches the technique used at block 404 may be used to determine the simulated signal, such as using the Scrappie squiggler tool.

At block 416, the molbit generation engine 312 determines pairwise distances between the simulated signal for the mutated barcode sequence and the other simulated signals. In some embodiments, the pairwise distances may be determined using a technique that matches the technique used at block 406.

The method 400 then proceeds to a decision block 418, where a determination is made regarding whether the pairwise distances have improved after the mutation. In some embodiments, pairwise distances may be considered to have been improved if both the minimum and the average pairwise distances have been improved. In some embodiments, additional determinations may be made at decision block 418 regarding whether the mutation has improved the overall set of barcode sequences. For example, a local variant of a Smith-Waterman (SW) algorithm may be used to determine pairwise sequence dissimilarities, and these sequence dissimilarities may be compared to a minimum dissimilarity threshold. Various constraints that affect the ability to synthesize, assemble, and measure the mutated barcode sequence may also be considered. For example, the mutated barcode sequence may be checked to see if it is within a range of allowed GC content (e.g., between 30-70% GC), has a maximum folding potential as determined using any suitable tool (e.g., a folding potential of −9 kcal/mol as calculated using NUPACK's MFE utility, other maximum folding potentials in a range from −8 kcal/mol to −10 kcal/mol), excludes any sequence used for other purposes (e.g., excludes the BsaI cut site sequence, GGTCTC), and/or has a maximum homopolymer length of five for A/T and four for C/G.

If the pairwise distances have not improved (or some other constraint is violated by the mutation), then the result of decision block 418 is NO, and the method 400 proceeds to block 420. At block 420, the molbit generation engine 312 reverts the mutation to the barcode sequence, and then returns to block 412 to attempt a different mutation to the barcode sequence. Otherwise, if the pairwise distances have improved (and any other relevant constraints are met), then the result of decision block 418 is YES, and the method 400 proceeds to the for-loop end block 422. In some embodiments, the method 400 may only return to block 412 from block 420 a limited number of times before proceeding on to for-loop end block 422. For example, in some embodiments, after returning to block 412 100 times, the method 400 may proceed to for-loop end block 422 from block 420 instead of returning to block 412.

At the for-loop end block 422, if further barcode sequences remain to be processed, then the method 400 loops back to for-loop start block 410 to process the next barcode sequence. Otherwise, if all of the barcode sequences have been processed, then the method 400 proceeds to decision block 424.

At decision block 424, a determination is made regarding whether the method 400 is done optimizing the set of barcode sequences. In some embodiments, the method 400 may perform a predetermined number of iterations, and may determine whether it is done based on whether the number of iterations has been reached. In some embodiments, the method 400 may continue to optimize the set of barcode sequences until the optimization begins bouncing between mutations to only two sequences. At such a point, the method 400 has produced a local minimum as the result of the series of random incremental improvements.

If the determination is that the method 400 is not done optimizing the set of barcode sequences, then the result of decision block 424 is NO, and the method 400 returns to block 408 to continue processing. Otherwise, if the method 400 is done optimizing the set of barcode sequences, then the result of decision block 424 is YES, and the method 400 proceeds to block 426. At block 426, the molbit generation engine 312 stores the set of barcode sequences. The set of barcode sequences may be stored in any suitable format, including but not limited to in a data store and/or on a computer-readable medium accessible by the molecular tagging computing system 302.

The method 400 then proceeds to an end block and terminates.

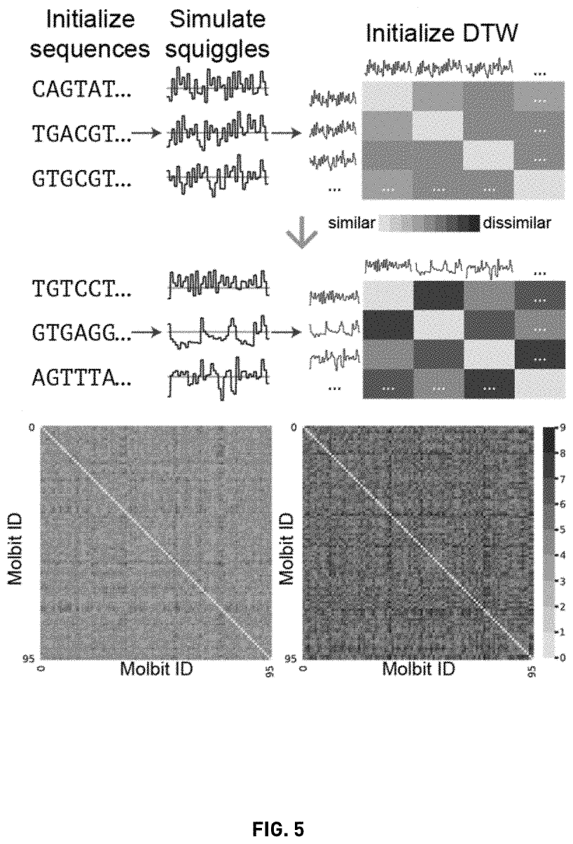

FIG. 5 is a chart that illustrates further aspects of a non-limiting example embodiment of the method described in FIG. 4. In the upper left, the set of barcode sequences is initialized, and simulated signals, or squiggles, are generated for each of the barcode sequences. In the upper-right, an illustration of the pairwise distances between the squiggles is shown, with darker colors indicating further distance. The middle-left illustrates an example of how the barcode sequences would appear after random mutations, and the middle-right illustrates the pairwise distances between the mutated barcode sequences. As can be seen, the colors are much darker, indicating a greater pairwise distance between each of the barcode sequences. The bottom-left and bottom-right illustrations also show pairwise distance increasing from the initial set of barcode sequences (left) to the mutated set of barcode sequences (right) on a larger set of 96 barcode sequences. In the illustrated embodiment, 31 iterations of the evolutionary model were executed. After initialization (left), the minimum DTW similarity was 2.9 (with a mean of 4.2+/−0.4), and after evolution (right), the minimum DTW similarity was 4.2 (with a mean of 5.8+/−0.8).

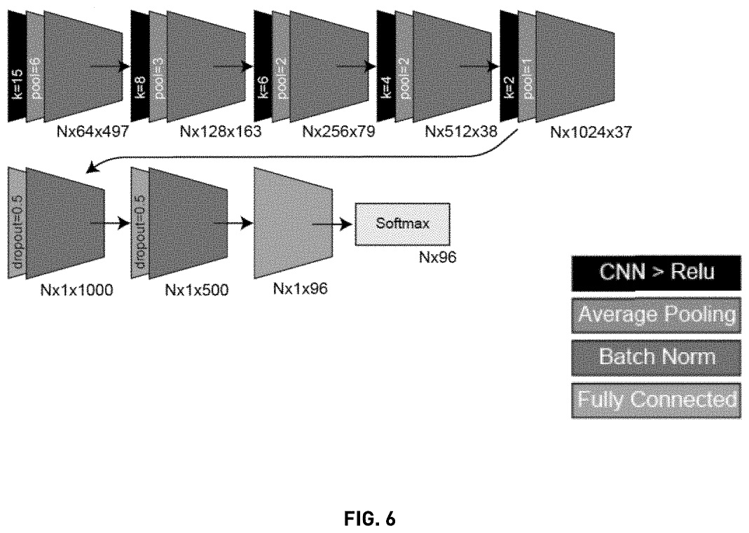

In some embodiments of the present disclosure, individual molbits are identified using a machine learning model which takes raw nanopore signals as input and provides an identity of a molbit as output with an associated confidence value. FIG. 6 is an illustration of a non-limiting example embodiment of a machine learning model suitable for use in identifying molbits according to various aspects of the present disclosure. As shown, the model includes a 5-layer convolutional neural network (CNN), followed by two fully connected layers with 50% dropout, and a final fully connected layer with softmax as the output layer. In the illustrated embodiment, each of the 5 CNN layers is identically structured, including a 1D convolutional layer with ReLU activation, average pooling, and then batch normalization. In some embodiments, various changes could be made to the architecture of this machine learning model while still accepting raw nanopore signals as input and providing identity of a molbit as output with an associated confidence value.

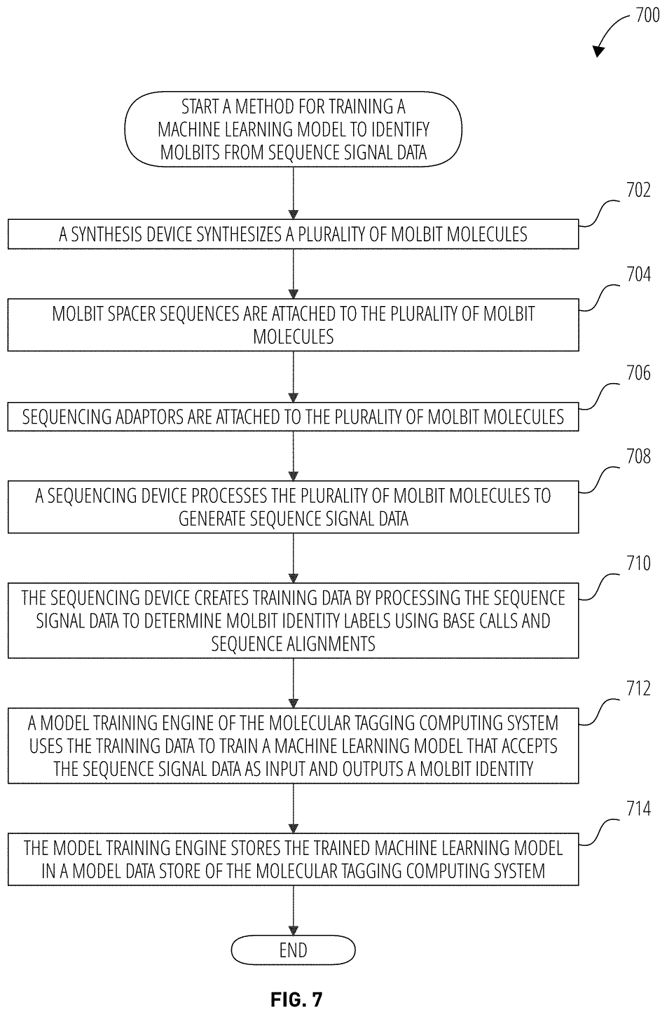

FIG. 7 is a flowchart that illustrates a non-limiting example embodiment of a method for training a machine learning model to identify molbits from sequence signal data according to various aspects of the present disclosure. The method 700 is an example of a technique suitable for training a machine learning model such as the one illustrated in FIG. 6 to recognize molbits such as those illustrated in FIG. 1.

From a start block, the method 700 proceeds to block 702, where a synthesis device 304 synthesizes a plurality of molbit molecules. In some embodiments, a training data set may be built by operating the synthesis device 304 to separately generate molbit molecules for each of the molbits, such that the molbit molecules may be stored and sequenced separately. In such embodiments, the identity of each molbit provided to the sequencing device 306 would be known. However, it can be cost-prohibitive to operate the synthesis device 304 in this manner for generating training data. Accordingly, in some embodiments, a training data set may be built by dividing the molbits into groups that may be synthesized together by the synthesis device 304. For example, a set of 96 molbits may be divided into 6 runs of 16 molbits each. The division may be made based on the molbits that have the highest predicted distances from each other, such that the molbits in each run may be accurately identified for the purposes of training. In some embodiments, the synthesis device 304 may synthesize forward and reverse strands of the barcode sequences. The reverse strands may contain a 5′ GATG overhang and a 3′ dA-tail. In some embodiments, the forward and reverse strands may be annealed by mixing them equimolar in a buffer solution such as 0.5M PBS, boiling them at about 94 C (e.g., at an appropriate temperature in a range from 92 C to 96 C) for two minutes and then allowing them to cool at room temperature. In some embodiments, different temperatures may be used, the concentrations may be different than equimolar, and different buffer solutions may be used.

At optional block 704, molbit spacer sequences are attached to the plurality of molbit molecules. In some embodiments, portions of a known plasmid may be cut and amplified in order to generate the spacer sequences. As one non-limiting example, an arbitrary 400 nt portion of plasmid pCDB180 may be amplified using PCR, and appropriate primers may be used to add BsaI cut sites to the ends of the amplified product. In some embodiments, the molbits may be assembled by ligating the desired annealed barcode sequence molecules with the spacer molecules. In some embodiments, ligation may be performed using a tool such as NEB's Golden Gate Assembly Kit.

At block 706, sequencing adaptors are attached to the plurality of molbit molecules. In some embodiments, sequencing adaptors appropriate for use with the specific sequencing device 306 being used may be attached. For example, if ONT's MinION device is used for sequencing, the molbit molecules may be prepared for sequencing using ONT's Ligation Sequencing Kit (SQK-LSK109) following the kit protocol and ONT's Flow Cell Priming Kit (EXP-FLP001).

At block 708, a sequencing device 306 processes the plurality of molbit molecules to generate sequence signal data. The sequence signal data generated by the sequencing device 306 is a raw nanopore signal that indicates ionic signals generated while the molbit molecules are processed by the device. As stated above, the sequencing device 306 may be ONT's MinION device. In such embodiments, the molbit molecules may be sequenced using a MinION flow cell with bulk FASTS raw data collection enabled on MinKNOW.

At block 710, the sequencing device 306 creates training data by processing the sequence signal data to determine molbit identity labels using base calls and sequence alignments. That is, the nucleotides read by the sequencing device 306 are matched up to the designed barcode sequences to determine the molbit identity labels for the training data. In some embodiments, the sequencing device 306 itself makes base call decisions and/or the sequence alignment decisions. In some embodiments, the model training engine 316 or other component of the molecular tagging computing system 302 makes the base calling decisions and performs the sequence alignments. In some embodiments, basecalling may be performed using a tool such as Guppy, and Smith-Waterman sequence alignment against the full set of barcode sequences may be used. In some embodiments, only training data in which the Smith-Waterman alignment score is greater than a predetermined threshold, including but not limited to values in a range from 13-17 (such as 15), may be used. In embodiments where each molbit is separately synthesized, performing these sequence alignments to label the training data may be unnecessary, because each set of molbit molecules could be kept physically separate.

At block 712, a model training engine 316 of the molecular tagging computing system 302 uses the training data to train a machine learning model that accepts the sequence signal data as input and outputs a molbit identity. As described above, the training data input to the machine learning model is the sequence signal data, and the labels for the training data are those determined at block 710. Any suitable technique for training the machine learning model, including but not limited to gradient descent, may be used. In some embodiments, the training data set may be pre-processed before being used for training. For example, the training data set may be arranged to have a similar number of read occurrences for each molbit. As another example, the raw sequence signal data may be scaled using a technique including but not limited to Median Absolute Deviation, the sequence signal data may be trimmed to remove a variable-length stalled signal characteristic to the beginning of sequencing reads, and the sequence signal may be truncated, such as to about the first 3000 data points. In some embodiments, training via gradient descent or any other suitable technique may be performed for a predetermined number of iterations.

In one test embodiment which trained the machine learning model of FIG. 6 for 108 iterations, the final training, validation, and test accuracy obtained were 99.93%, 97.70%, and 96.96%, respectively, compared to the sequence-derived labels. What is more, when the trained machine learning model was applied to all of the sequence signal information (not just the sequence signal information that was successfully labeled using base calling and sequence alignment), the machine learning model was able to confidently classify 97% of the reads in the test set, compared to 75.1% of the reads in the test set by base calling and sequence alignment.

At block 714, the model training engine 316 stores the trained machine learning model in a model data store 310 of the molecular tagging computing system 302.

The method 700 then proceeds to an end block and terminates.

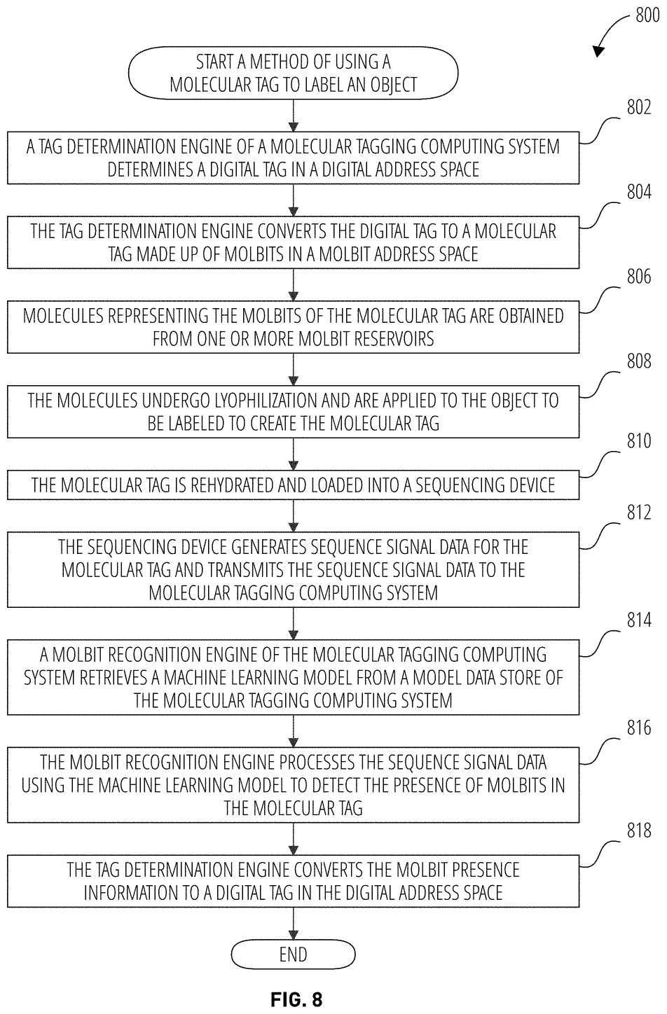

FIG. 8 is a flowchart that illustrates a non-limiting example embodiment of a method of using a molecular tag to label an object according to various aspects of the present disclosure. Once the molbits are designed and synthesized, the method 800 can be used to label objects without relying on sophisticated synthesis hardware and without requiring the time and expense of full sequence-alignment based identification of the molbits in the label.

From a start block, the method 800 proceeds to block 802, where a tag determination engine 320 of a molecular tagging computing system 302 determines a digital tag in a digital address space. At block 804, the tag determination engine 320 converts the digital tag to a molecular tag made up of molbits in a molbit address space. The simplest method for encoding information in a molecular tag is a naive 1:1 mapping between digital bits and molbits. However, with this method, even a single bit error makes the tag unrecoverable (that is, it produces an incorrect decoding).

Since a reliable tagging system should have a very low chance of incorrect decoding (e.g., 1 in 1 billion), some embodiments of the present disclosure use a digital address space that is smaller than the molbit address space so that error correcting codes (ECCs) may be inserted in the molecular tag. The molecular tag uses the presence or absence of reads for a given molbit to encode information. ECCs reduce the possibility of unrecoverable tags despite the possibility of a high per-bit error rate by creating a codeword by projecting a message from the digital address space into the larger molbit address space with greater separability. This allows more bits to be incorrectly decoded before the message is decoded incorrectly.

Any suitable technique for adding an error correcting code while projecting the tag in the digital address space to the molbit address space may be used. In some embodiments, the message in the digital address space may be multiplied by a binary matrix of random numbers, known as a random generator matrix, to project the message into the molbit address space. A digital address space of 32 bits may provide ˜4.2 billion total unique tags, and when projected into a 96 molbit address space using a random generator matrix, produces a 1.6×10\hat{ }−11 chance of incorrect decoding, even at an error rate of 1.70%.

At block 806, molecules representing the molbits of the molecular tag are obtained from one or more molbit reservoirs 308. In the molecular tag, presence of a given molbit molecule represents a “1” in the molbit address space, and absence of a given molbit molecule represents a “0” in the molbit address space. Accordingly, once the digital tag is projected into the molbit address space, equal amounts of each molbit associated with a “1” in the molbit address space are obtained from the molbit reservoirs 308 and mixed together. Any suitable technique, including but not limited to automatic or manual pipetting, may be used to obtain the amounts of each molbit from the molbit reservoirs 308. In some embodiments, after being mixed, the combined molbits may be prepared for sequencing using a technique similar to those discussed above in block 706, including but not limited to attaching sequencing adapters appropriate for use with the sequencing device 306.

At block 808, the molecules undergo lyophilization and are applied to the object to be labeled to create the molecular tag. Any suitable technique may be used for lyophilizing the sample. For example, in some embodiments, the molbit molecules may first be mixed with 1% trehalose dihydrate solution before being lyophilized. The lyophilized molecules may be applied to the object using any suitable technique, including but not limited to painting and fogging.

The lyophilized molecules have been shown to be particularly stable. In one example test, a molecular tag was shipped via regular mail from Seattle, Wash. to Santa Cruz, Calif., where it sat at room temperature for 4-5 weeks before being successfully analyzed as described below.

At block 810, the molecular tag is rehydrated and loaded into a sequencing device 306. Any suitable technique may be used to rehydrate the molecular tag, including but not limited to applying water or another buffer solution to the molecular tag (or, in general, to the tagged object).

At block 812, the sequencing device 306 generates sequence signal data for the molecular tag and transmits the sequence signal data to the molecular tagging computing system 302. At block 814, a molbit recognition engine 318 of the molecular tagging computing system 302 retrieves a machine learning model from a model data store 310 of the molecular tagging computing system 302. The machine learning model is a machine learning model that was trained on the same information used to generate the molbits in the molbit reservoirs 308 using a method similar to method 700 described above.

At block 816, the molbit recognition engine 318 processes the sequence signal data using the machine learning model to detect the presence of molbits in the molecular tag. In some embodiments, the machine learning model outputs molbit identities for each molbit molecule that it processes. In some embodiments, the molbit recognition engine 318 may use the sequence signal data to identify the barcode sequence represented by a read, and may use a length of the read to identify which specific molbit (amongst different molbits that use the same barcode sequence but different-length spacer sequences) is represented.

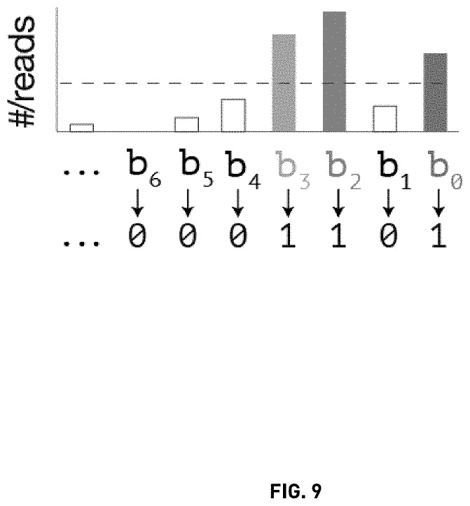

The molbit recognition engine 318 may tally each molbit identification as the molbit molecules are processed. FIG. 9 is a chart that illustrates tallies of various molbits identified by the machine learning model according to various aspects of the present disclosure. As shown, each time the molbit recognition engine 318 identifies a given molbit, a tally is added for the given molbit. Once the total number of reads reaches a predetermined threshold for a given molbit, that molbit is considered present in the molecular tag. As shown in FIG. 9, molbits b₀, b₂, and b₃ have crossed the predetermined threshold and are considered present, while the remainder of the molbits are considered absent. In some embodiments, the predetermined threshold may be adjusted based on processing of the error correcting codes.

Returning to FIG. 8, at block 818, the tag determination engine 320 converts the molbit presence information to a digital tag in the digital address space. The conversion of the molbit presence information to the digital tag is an inverse of the conversion performed in block 804. For example, in an embodiment that used a multiplication by a binary matrix of random numbers to convert the tag from the digital address space to the molbit address space, the tag may be converted back from the molbit address space to the digital address space using brute-force nearest neighbor decoding.

The method 800 then proceeds to an end block and terminates.

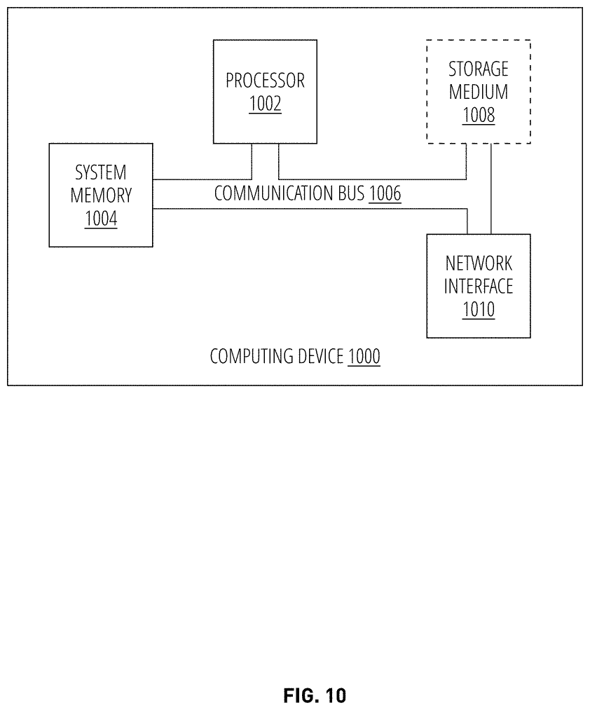

FIG. 10 is a block diagram that illustrates aspects of an exemplary computing device 1000 appropriate for use as a computing device of the present disclosure. While multiple different types of computing devices were discussed above, the exemplary computing device 1000 describes various elements that are common to many different types of computing devices. While FIG. 10 is described with reference to a computing device that is implemented as a device on a network, the description below is applicable to servers, personal computers, mobile phones, smart phones, tablet computers, embedded computing devices, and other devices that may be used to implement portions of embodiments of the present disclosure. Some embodiments of a computing device may be implemented in or may include an application-specific integrated circuit (ASIC), a field-programmable gate array (FPGA), or other customized device. Moreover, those of ordinary skill in the art and others will recognize that the computing device 1000 may be any one of any number of currently available or yet to be developed devices.

In its most basic configuration, the computing device 1000 includes at least one processor 1002 and a system memory 1004 connected by a communication bus 1006. Depending on the exact configuration and type of device, the system memory 1004 may be volatile or nonvolatile memory, such as read only memory (“ROM”), random access memory (“RAM”), EEPROM, flash memory, or similar memory technology. Those of ordinary skill in the art and others will recognize that system memory 1004 typically stores data and/or program modules that are immediately accessible to and/or currently being operated on by the processor 1002. In this regard, the processor 1002 may serve as a computational center of the computing device 1000 by supporting the execution of instructions.

As further illustrated in FIG. 10, the computing device 1000 may include a network interface 1010 comprising one or more components for communicating with other devices over a network. Embodiments of the present disclosure may access basic services that utilize the network interface 1010 to perform communications using common network protocols. The network interface 1010 may also include a wireless network interface configured to communicate via one or more wireless communication protocols, such as Wi-Fi, 2G, 3G, LTE, WiMAX, Bluetooth, Bluetooth low energy, and/or the like. As will be appreciated by one of ordinary skill in the art, the network interface 1010 illustrated in FIG. 10 may represent one or more wireless interfaces or physical communication interfaces described and illustrated above with respect to particular components of the computing device 1000.

In the exemplary embodiment depicted in FIG. 10, the computing device 1000 also includes a storage medium 1008. However, services may be accessed using a computing device that does not include means for persisting data to a local storage medium. Therefore, the storage medium 1008 depicted in FIG. 10 is represented with a dashed line to indicate that the storage medium 1008 is optional. In any event, the storage medium 1008 may be volatile or nonvolatile, removable or nonremovable, implemented using any technology capable of storing information such as, but not limited to, a hard drive, solid state drive, CD ROM, DVD, or other disk storage, magnetic cassettes, magnetic tape, magnetic disk storage, and/or the like.

Suitable implementations of computing devices that include a processor 1002, system memory 1004, communication bus 1006, storage medium 1008, and network interface 1010 are known and commercially available. For ease of illustration and because it is not important for an understanding of the claimed subject matter, FIG. 10 does not show some of the typical components of many computing devices. In this regard, the computing device 1000 may include input devices, such as a keyboard, keypad, mouse, microphone, touch input device, touch screen, tablet, and/or the like. Such input devices may be coupled to the computing device 1000 by wired or wireless connections including RF, infrared, serial, parallel, Bluetooth, Bluetooth low energy, USB, or other suitable connections protocols using wireless or physical connections. Similarly, the computing device 1000 may also include output devices such as a display, speakers, printer, etc. Since these devices are well known in the art, they are not illustrated or described further herein.

While illustrative embodiments have been illustrated and described, it will be appreciated that various changes can be made therein without departing from the spirit and scope of the invention.

What is claimed is:

1. A system, comprising:
a sequencing device configured to generate signals based upon sequences of nucleic acid molecules;
a plurality of nucleic acid molecules, wherein each nucleic acid molecule in the plurality of nucleic acid molecules includes a barcode sequence associated with a molbit of a set of molbits; and
a computing system communicatively coupled to the sequencing device, the computing system including logic that, in response to execution by at least one processor of the computing system, causes the computing system to perform actions for determining a digital tag represented by the plurality of nucleic acid molecules, the actions comprising:
receiving, from the sequencing device, a plurality of signals, wherein signals of the plurality of signals represent raw nanopore signals detected by the sequencing device while sequencing at least the barcode sequences of the plurality of nucleic acid molecules;
identifying molbits represented by at least the barcode sequences of the plurality of nucleic acid molecules based on the plurality of signals; and
determining a digital tag based on the identified molbits.

2. The system of claim 1, wherein each molbit is associated with a barcode sequence designed to generate a signal output from the sequencing device with a maximum pairwise dynamic time warping (DTW) distance from signal outputs from the sequencing device generated by other barcode sequences associated with other molbits.

3. The system of claim 2, wherein the barcode sequences are designed using an iterative mutation technique.

4. The system of claim 1, wherein identifying molbits represented by at least the barcode sequences of the plurality of nucleic acid molecules based on the plurality of signals includes, for each signal of the plurality of signals:
providing the signal to a machine learning model to determine a molbit represented by the signal.

5. The system of claim 4, wherein identifying molbits represented by at least the barcode sequences of the plurality of nucleic acid molecules based on the plurality of signals further includes, for each molbit of the set of molbits:
tallying a number of signals that represent the molbit;
in response to determining that the number of signals is greater than a predetermined threshold, indicating that the molbit is present in the plurality of nucleic acid molecules; and
in response to determining that the number of signals is not greater than the predetermined threshold, indicating that the molbit is absent in the plurality of nucleic acid molecules.

6. The system of claim 5, wherein determining the digital tag based on the identified molbits includes converting the indications of present molbits and absent molbits to a digital address space.

7. The system of claim 6, wherein determining the digital tag based on the identified molbits includes performing error correction to convert the indications of present molbits and absent molbits to the digital address space.

8. The system of claim 1, wherein each molbit is associated with a spacer sequence having a first length or a second length.

9. The system of claim 8, wherein identifying molbits represented by at least the barcode sequences of the plurality of nucleic acid molecules based on the plurality of signals includes, for each signal of the plurality of signals:
providing the signal to a machine learning model to determine a barcode sequence represented by the signal; and
determining a molbit represented by the signal based on the barcode sequence and a read length of the signal.

10. A kit for tagging objects with molecular tags that represent digital tag values, the kit comprising:
a plurality of molbit reservoirs, wherein each molbit reservoir is associated with a molbit and includes nucleic acid molecules that represent the molbit.

11. The kit of claim 10, wherein each nucleic acid molecule includes a barcode sequence, and wherein the barcode sequence for each molbit is designed based on maximizing a pairwise dynamic time warping (DTW) distance between a sequencing trace signal for the barcode sequence and sequencing trace signals of the other barcode sequences.

12. The kit of claim 11, wherein each nucleic acid molecule includes a spacer sequence, and wherein each molbit is associated with either a spacer sequence of a first length or a spacer sequence of a second length.

13. The kit of claim 11, wherein each nucleic acid molecule includes a sequencing adapter.

14. The kit of claim 10, further comprising a computing system having logic stored therein that, in response to execution by at least one processor of the computing system, causes the computing system to:
determine a digital tag value;
convert the digital tag value to a molbit tag value; and
generate instructions for creating a molecular tag having the molbit tag value by combining nucleic acid molecules from the plurality of molbit reservoirs.

15. The kit of claim 14, wherein a binary length of the digital tag value is smaller than a number of molbit reservoirs in the plurality of molbit reservoirs.

16. The kit of claim 15, wherein converting the digital tag value to the molbit tag value includes determining one or more error correction values to include in the molbit tag value.

17. A method of tagging an object with a molecular tag, the method comprising:
determining a digital tag value;
converting the digital tag value to a molbit tag value, the molbit tag value indicating presence and absence of a plurality of molbit values;
combining nucleic acid molecules associated with each molbit value indicated as present in the molbit tag value; and
applying the combined nucleic acid molecules to the object.

18. The method of claim 17, further comprising lyophilizing the combined nucleic acid molecules before application to the object.

19. The method of claim 18, further comprising rehydrating the lyophilized combined nucleic acid molecules that were applied to the object;
providing the rehydrated combined nucleic acid molecules to a sequencing device;
obtaining sequence signal data from the sequencing device, the sequence signal data including nanopore trace information;
identifying the molbit values associated with the combined nucleic acid molecules based on the nanopore trace information; and
determining the digital tag value based on the identified molbit values.

20. The method of claim 19, wherein the sequence signal data further includes read length information, and wherein identifying the molbit values associated with the combined nucleic acid molecules is further based on the read length information.
